(19) United States
(12) Patent Application Publication
(10) Pub. No.: US 2004/0096864 A1
(43) Pub. Date: May 20, 2004

(54) Method of eukaryotic expression cloning of disease associated molecules

(76) Inventors: Miles W. Carroll, Oxford (GB); Susan M. Kingsman, Oxford (GB)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/470,363

(22) PCT Filed: Feb. 6, 2002
(86) PCT No.: PCT/GB02/00531

(30) Foreign Application Priority Data
Feb. 6, 2001 (GB) 0102947.9

(51) Int. Cl. C12Q 1/68; C12N 15/86

(52) U.S. Cl. 435/6; 435/456

(57) ABSTRACT

The present invention describes a method for identifying a target disease associated molecule (DAM) comprising: generating an expression library from DNA or RNA derived from a cell expressing the DAM; transfecting the library into a eukaryotic host cell; and screening the expression library with a screen comprising a binding partner to identify DNA clones expressing the target DAM. The library may produced by inserting the cDNA library into a virus based vector, such as a pox viral vector with a minimal viral genome.

### FIELD OF THE INVENTION

The present invention relates to a method.

In particular, the present invention relates to a method for identifying a disease associated molecule (DAM).

More in particular, the present invention relates to a method for identifying a target DAM by expressing an expression library in a eukaryotic system.

Even more in particular, the present invention relates to the application of an identified DAM in the diagnosis and treatment of diseases associated with a DAM.

### BACKGROUND OF THE INVENTION

In certain disease states, a derangement of cellular metabolism can affect the level of expression of one or more DAMs. In some circumstances, this cellular derangement may lead to a change in the levels of expression of the DAM. Thus, each disease causing agent or disease state may have associated with it a DAM which may be crucial in the immune recognition and/or the elimination and/or control of a disease causing agent or disease state in a host organism. In this way, the DAM may be capable of acting as a marker not only for the diagnosis of disease states but also for the accurate staging of the disease profile so that the appropriate therapy may be designed.

A particular example of DAMs which have been well characterised include the tumour-associated antigens (TAAs). A number of oncofoetal or tumour-associated antigens (TAAs) have been identified and characterised in human and animal tumours.

These TAAs include carcinoembryonic antigen (CEA), -TAG72, c-erB2, (underglycosylated) MUC-1 and p53, epithelial glycoprotein-2 antigen (EGP-2; also known as EGP40, Ep-CAM, KSA, CO17-1A or GA733-2) and the 5T4 antigen. In general, TAAs are antigens which are expressed during foetal development but which are downregulated in adult cells, and are thus normally absent or present only at very low levels in adults. However, during tumourigenesis, tumour cells have been observed to resume expression of TAAs. Thus, it is thought that malignant cells may be distinguished from their non-malignant counterparts by resumption of expression of TAAs. Consequently, application of TAAs for (i) in vitro and/or in vivo/ex vivo diagnosis of tumour disorders; (ii) for imaging and/or immunotherapy of cancer has been suggested and (iii) as indicators of progression of tumour associated disease;

It is well known that during natural tumour cell degradation in vivo, the host can mount an immune response to proteins over expressed in or on the cancer cells. The intensity of such responses can increase as the cancer progresses. That is, serum from late stage cancer patients may have more intense anti-TAA antibody responses.

A method, called serological analysis of autologous tumour antigens by recombinant cDNA expression cloning (hereinafter called SEREX) has been developed by Tureci et al (1997) for the identification of target tumour associated antigens (TAAs) in which a tumour cell expression library is constructed in a bacterial virus based vector such as lambda phage vector. Using this method, the contents of the library are expressed in E. coli which are are then transferred on to nitrocellulose membranes and permeabilised. Serum from cancer patients (usually from the same patient that the tumour library was derived from) is used to identify TAAs using an immunoscreen technique. Many hundreds of potential TAAs have been identified using this approach.

However, a major disadvantage with the SEREX method is that it relies on prokaryotic expression of the potential TAA. Unlike eukaryotic cells which are well known for their ability to post-translationally modify proteins, prokaryotic cells do not carry out these modifications to the protein and therefore many conformational epitopes are not present. Thus, using the SEREX method, antibodies will not recognise epitopes whose conformation is reliant on specific post-translational modification that are unique to the eukaryotic expression system. By way of example, SEREX will not be able to identify those TAA in which the natural cancer patient immune response is mounted against conformation epitopes that are reliant on authentic post-translational modifications such as glycosylation. Examples of glycosylated TAAs include but are not limited to CEA, 5T4, EpCAM, and MUC-1 antigens.

Additionally, expression on the cell surface may also effect the folding and the three dimensional configuration of the target DAM which will not be authentically expressed in the SEREX system as outlined above.

The development of a eukaryotic expression system for identifying target DAMs is not a staightforward undertaking for at least two reasons: These are:

(i) the transfection of plasmid cDNA libraries into mammalian cells is relatively inefficient when compared to the transfection of bacteriophage libraries into prokaryotic cells; and

(ii) a sensitive screening system is required to identify the potential target antigen and which does not kill the cell.

One screening process known in the art uses a plasmid cDNA library made from a tumour line known to be lysed by a cytotoxic T lymphocyte (CTL) cell line. This approach was devised by Boone et at., 1994 (Annual Reviews in Immunology 12: 337-366). Pools of cDNA in plasmid form are transfected into target cells (expressing the relevant MHC type for the corresponding CTL). CTL lines are then incubated with the transfected cells. CTL killing of the target cell occurs if the target cDNA is contained within the transfected pool. CTL activity can be measured by cytokine release. The cDNA pool is further dissected to identitify the individual cDNA. However, his technique suffers from the disadvantage that it is extremely time consuming and a source of patient derived tumour specific CTLs is required. Additionally, the the cDNA library must have sequences within it that can activate and be recognised by CTLs. Screening of the library also relies on DNA transfection techniques which have varying efficiencies.

Another method described in WO 00/28016 is used to enrich for, and select for those cells infected with the recombinant viruses that express the target epitopes of specific cytotoxic T cells. An adherent monolayer of cells is infected with a recombinant viral library, such as a vaccinia recombinant viral library. It is important that these cells do not themselves express the target epitopes recognized by specific CTLs but that these epitopes are represented in the viral library. In addition, for selection by CTLs, the infected cells must express an appropriate MHC molecule that can associate with and present the target peptide to T cells. After infection with recombinant virus, the monolayer is washed to remove any non-adherent cells and CTLs of defined specificity are added. During this time, some of the adherent cells infected with a recombinant particle that leads to expression of the target epitope will interact with a specific CTL and undergo a lytic event. Cells that undergo a lytic event are released from the monolayer and can be harvested in the floating cell population. The above-described protocol is repeated for preferably five or more cycles, to increase the level of enrichment obtained by this procedure.

However, his technique suffers from the disadvantage that the cDNA library must have sequences within it that can activate and be recognised by CTLs.

The present invention seeks to provide an improved approach to the identification of target DAMs by using an improved expression library and an improved screening system.

### DETAILED ASPECTS OF THE PRESENT INVENTION

Aspects of the present invention are presented in the accompanying claims and in the following description and drawings. These aspects are presented under separate section headings. However, it is to be understood that the teachings under each section are not necessarily limited to that particular section heading.

Advantages

The present invention is advantageous because:

(i) the eukaryotic expression system enables the identification of post-translationally modified DAM;

(ii) the eukaryotic expression system enables the identification of natural oligomeric forms of proteins especially if the identifying screen comprises a binding partner which is specific to conformational epitopes that are unique to oligomers of the DAM;

(iii) additionally, the DAM will undergo authentic presentation on the cell surface (for review see Carroll et al 2001).

(iv) Vaccinia virus (VV) can replicate and therefore amplification of the target virus is possible. Direct transfection of plasmids, carrying cDNA libraries do not have this ability so the cDNA needs to be “rescued” from a single cell. If cDNA is to be rescued from a cell, it is essential to keep the cell alive if that is the only route of amplification for the cDNA. If the gene product of interest is toxic to the cell, it is extremely difficult to amplify the cDNA source by cell replication. In contrast, for VV, even if the cell product is toxic, the virus will still under go limited replication;

(v) VV has an extremely broad host range i.e. it replicates in an extremely diverse number of different cell types at very high efficiencies. Certain cell types are very poorly transfected. Moreover, VV promoters have very high expression levels. These are said to be significantly higher that CMV or LTR promoters. In contrast, although retroviral vectors have been used as library vectors, their efficacy of transduction of different cell types is very variable. In addition, retroviral vectors such as MLV, do not transduce non-replicating cells;

(vi) unlike the known methods for detecting the expressed DAM where the cells have to be lysed, the viral cells of the present invention can be maintained alive. If necessary, the virally infected cells can be perforate which allow for the staining of internally expressed antigens but which still keeps the virus alive.

Other advantages are discussed and made apparent in the following commentary.

Disease Associated Molecule (DAM)

As used herein, the term “DAM” can include but is not limited to biological response modifiers which include but are not limited to immunomodulators, cytokines, growth factors, cell surface receptors, hormones, circulatory molecule, inflammatory cytokines, and pathogenic agents such a viruses, bacteria, parasites or yeast. Examples of these biological response modifiers include but are not limited to ApoE, Apo-SAA, BDNF, Cardiotrophin-1, EGF, ENA-78, Eotaxin, Eotaxin-2, Exodus-2, FGF-acidic, FGF-basic, fibroblast growth factor-10 (Marshall 1998 Nature Biotechnology 16: 129), FLT3 ligand (Kimura et al. (1997), Fractalkine (CX3C), GDNF, G-CSF, GM-CSF, GF-β1, insulin, IFN-γ, IGF-I, IGF-II, IL-1α, IL-1β, IL-2, IL-3, IL-4, IL-5, IL-6, IL-7, IL-8 (72 a.a.), IL-8 (77 a.a.), IL-9, IL-10, IL-11, IL-12, IL-13, IL-15, IL-16, IL-17, IL-18 (IGIF), Inhibin α, Inhibin β, IP-10, keratinocyte growth factor-2 (KGF-2), KGF, Leptin, LIF, Lymphotactin, Mullerian inhibitory substance, monocyte colony inhibitory factor, monocyte attractant protein (Marshall 1998 ibid), M-CSF, MDC (67 a.a.), MDC (69 a.a.), MCP-1 (MCAF), MCP-2, MCP-3, MCP-4, MDC (67 a.a.), MDC (69 a.a.), MIG, MIP-1α, MIP-1β, MIP-3α, MIP-3β, MIP-4, myeloid progenitor inhibitor factor-1 (MPIF-1), NAP-2, Neurturin, Nerve growth factor, α-NGF, NT-3, NT-4, Oncostatin M, PDGF-AA, PDGF-AB, PDGF-BB, PF-4, RANTES, SDF1α, SDF1β, SCF, SCGF, stem cell factor (SCF), TARC, TGF-α, TGF-β, TGF-β2, TGF-β3, tumour necrosis factor (TNF), TNF-α, TNF-β, TNIL-1, TPO, VEGF, GCP-2, GRO/MGSA, GRO-β and GRO-γ.

Examples of pathogenic agents can include but are not limited to viruses, bacteria and parasites and yeasts. By way of example, pathogenic viruses include but are not limited to human immunodeficiency virus (HIV), influenza, herpes simplex, human papilloma virus, equine encephalitis virus, hepatitis, feline leukaemia virus, canine distemper and rabies virus, influenza, poxviruses, fowl pox virus (FPV), canarypox virus, entomopox virus, vaccinia virus deficient in a DNA replication enzyme, Alphavirus, adenovirus, herpesvirus, Venezuelan equine encephalitis virus (VEE). Examples of pathogenic bacteria can include but are not limited to Chlamydia, Mycobacteria, Legioniella, Staphilococcus, S. aureus, Helicobacter, Campylobacter, Shigella, Brucella, Pseudomonas aeruginosa, Salmonella typhimurium, Streptococcus pyogenes, Neisseria onorrheae, Corynebacterium diphtheriae, Clostridium tetani, Vibrio cholerae, Vibrio arahaemoliticus, Listeria monocytogenes, Clostridium perfringens, Escherichia coli, Yersinia pestis, Streptococcus pneumoniae and S. typhimurium. Examples of pathogenic arasites include but are not limited to Plasmodium Falciparum, Trypanosoma, Trypanosoma cruzi, Leishmania, Leishmania ddnovani, L. tropica, L. mexicana, L. braziliensis, Giardia, Giardia lamblia, Trichomonas, Entamoeba, Naegleria, Acanthamoeba, Acanthamoeba castellanii, A. culbertsoni and other species, Plasmodium, Plasmodium falciparum, Toxoplasma, Toxoplasma gondii, Cryptosporidium, Cryptosporidium parvum, Isospora, Isospora belli, Naegleria, Naegleria fowleri, Balantidium, Balantidium coli, Babesia, Schistosoma, Toxiplasma and Toxocara canis. Examples of pathogenic yeasts include certain species of Aspergillus and invasive Candida. In a preferred embodiment the pathogenic microorganism is an intracellular organism.

Preferably the DAM is an intracellular pathogenic agent.

Preferably the DAM is a disease associated cell surface molecule (DACSM).

In accordance with the present invention the DACSM can include but is not limited to a receptor for adhesive proteins such as growth factor receptors. Examples of growth factor receptors include but are not limited to ApoE, Apo-SAA, BDNF, Cardiotrophin-1, EGF, ENA-78, Eotaxin, Eotaxin-2, Exodus-2, FGF-acidic, FGF-basic, fibroblast growth factor10 (Marshall 1998 Nature Biotechnology 16: 129) FLT3 ligand (Kimura et al (1997), Fractalkine (CX3C), GDNF, G-CSF, GM-CSF, GF-β1, insulin, IFN-γ, IGF-I, IGF-II, IL-1α, IL-1β, IL-2, IL-3, IL-4, IL-5, IL-6, IL-7, IL-8 (72 a.a.), IL-8 (77 a.a.), IL-9, IL-10, IL-11, IL-12, IL-13, IL-15, IL-16, IL-17, IL-18 (IGIF), Inhibin a, Inhibin p, IP-10, keratinocyte growth factor-2 (KGF-2), KGF, Leptin, LIF, Lymphotactin, Mullerian inhibitory substance, monocyte colony inhibitory factor, monocyte attractant protein (Marshall 1998 ibid), M-CSF, MDC (67 a.a.), MDC (69 a.a.), MCP-1 (MCAF), MCP-2, MCP-3, MCP-4, MDC (67 a.a.), MDC (69 a.a.), MIG, MIP-1α, MIP-1β, MIP-3α, MIP-3β, MIP-4, myeloid progenitor inhibitor factor-1 (MPIF-1), NAP-2, Neurturin, Nerve growth factor, β-NGF, NT-3, NT-4, Oncostatin M, PDGF-AA, PDGF-AB, PDGF-BB, PF-4, RANTES, SDF1α, SDF1β, SCF, SCGF, stem cell factor (SCF), TARC, TGF-α, TGF-β, TGF-β2, TGF-β3, tumour necrosis factor (TNF), TNF-α, TNF-β, TNIL-1, TPO, VEGF, GCP-2, GRO/MGSA, GRO-β, GRO-γ, HCC1, 1-309. A non-exhaustive list of growth factor receptors can be found on pages 392-297 Molecular Biology and Biotechnology (Ed R A Meyers 1995 VCH Publishers Inc).; a plasminogen activator; a metalloproteinase (such as colllagenase), a mucin; a glycoprotein; an antigen restricted in its tissue distribution; and/or a cell surface molecule which plays a role in tumour cell growth, migration or metastasis, (such as a 5T4 antigen, a tumour specific carbohydrate moiety or an oncofetal antigen). The term DACSM may also includes antigenic determinants.

Antigenic Determinant

As used herein, the term “antigenic determinant” refers to any antigen which is associated with a disease or a disorder. By way of example, the antigenic determinant may also be derived from pathogenic agents associated with diseased cells, such as tumour cells, which multiply unrestrictedly in an organism and may thus lead to pathological growths. Examples of such pathogenic agents are described in Davis, B. D. et al., (Microbiology, 3rd ed., Harper International Edition). The antigenic determinant may be an antigen and/or an immunodominant epitope on an antigen. By way of example, the antigenic determinant may include tumour associated antigens (TAA) which may serve as targets for the host immune system and elicit responses which result in tumour destruction.

TAA

The term “tumour associated antigen (TAA)” is used herein to refer to any TAA or antigenic peptide thereof. The antigen being one that is expressed by the tumour itself or cells associated with the tumour such as parenchymal cells or those of the associated vasculature. The term “tumour associated antigen (TAA)” includes antigens that distinguish the tumour cells from their normal cellular counterparts where they may be present in trace amounts.

Examples of TAAs include but are not limited to MART-1 (Melanoma Antigen Recognised by T cells-1) MAGE-1, MAGE-3, 5T4, gp100, Carcinoembryonic antigen (CEA), prostate-specific antigen (PSA), MUCIN (MUC-1), tyrosinase. Particularly preferred TAAs are cell surface molecules as these are positioned for recognition by elements of the immune system and are excellent targets for therapy such as therapy and/or immunotherapy. The present invention is in no way limited to antigenic determinants encoding the above listed TAAs. Other TAAs may be identified, isolated and cloned by methods known in the art such as those disclosed in U.S. Pat. No. 4,514,506.

5T4 TAA

The TAA 5T4 (see WO 89/07947) has been extensively characterised. It is a 72 kDa glycoprotein expressed widely in carcinomas, but having a highly restricted expression pattern in normal adult tissues. It appears to be strongly correlated to metastasis in colorectal and gastric cancer. The full nucleic acid sequence of human 5T4 is known (Myers et al., 1994 J Biol Chem 169: 9319-24).

cDNA Library Generation

Cells are chosen to prepare a library of complementary DNA (that is, “cDNA”). These cells include but are not limited to normal cells or cells from a subject afflicted with a pathological condition which are exemplary of the pathological condition. By way of example, if the subject has melanoma, the cells are melanoma cells. If the subject is suffering from a neural disorder, then the cells are preferably a sample of the afflicted cells. This approach is chosen because the afflicted cells are most probably the best source of DAMs. That is, such molecules which are specifically associated with the pathological condition of interest. As indicated above, the term “DAM” may include but are not limited to antigenic determinants or for example, receptor molecules for specific ligands.

The preparation of the expression library is based upon the established fact that if proteins are expressed by the cells, then messenger RNA (mRNA) must be present. These mRNA molecules are not long lived, and are unstable, so they are not practical to work with. Accordingly, the cells chosen are then used to prepare a library of complementary DNA (i.e., “cDNA”). cDNA is first prepared from messenger RNA isolated from the cell by reverse transcription. Protocols for the generation of cDNA libraries through reverse transcription of mRNA sequences are well known in the art and kits for doing so are commercially available (from Gibco BRL, for instance). Once the cDNA is made, it is used to construct a vector library. In short, carrier vectors are treated, such as by cutting and splicing, to receive molecules of cDNA. The choice of vector may vary, as the skilled person is well familiar with many such examples.

Vector

As it is well known in the art, a vector is a tool that allows or faciliates the transfer of an entity from one environment to another. In accordance with the present invention, and by way of example, some vectors used in recombinant DNA techniques allow entities, such as a segment of DNA (such as a heterologous DNA segment, such as a heterologous cDNA segment), to be transferred into a host cell for the purpose of replicating the vectors comprising the nucleotide sequences of the present invention and/or expressing target DAMs of the present invention encoded by the nucleotide sequences of the present invention. Examples of vectors used in recombinant DNA techniques include but are not limited to plasmids, chromosomes, artificial chromosomes or viruses.

The term “vector” includes expression vectors and/or transformation vectors.

The term “expression vector” means a construct capable of in vivo or in vitrolex vivo expression.

The term “transformation vector” means a construct capable of being transferred from one species to another.

Preferably the vector is a virus based vector.

Viral Vectors

The vectors comprising cDNA nucleotide sequences of the present invention may be introduced into suitable host cells using a variety of viral techniques which are known in the art, such as for example infection with recombinant viral vectors such as retroviruses, herpes simplex viruses and adenoviruses.

Preferably the vector is a recombinant viral vectors. Suitable recombinant viral vectors include but are not limited to adenovirus vectors, adeno-associated viral (AAV) vectors, herpes-virus vectors, a retroviral vector, lentiviral vectors, baculoviral vectors, pox viral vectors or parvovirus vectors (see Kestler et al., 1999 Human Gene Ther 10(10):1619-32). In the case of viral vectors, gene delivery is mediated by viral infection of a target cell.

Retroviral Vectors

Examples of retroviruses include but are not limited to: murine leukemia virus (MLV), human immunodeficiency virus (HIV), equine infectious anaemia virus (EIAV), mouse mammary tumour virus (MMTV), Rous sarcoma virus (RSV), Fujinami sarcoma virus (FuSV), Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus-29 (MC29), and Avian erythroblastosis virus (AEV).

Preferred vectors for use in accordance with the present invention are recombinant viral vectors, in particular recombinant retroviral vectors (RRV) such as lentiviral vectors.

The term “recombinant retroviral vector” (RRV) refers to a vector with sufficient retroviral genetic information to allow packaging of an RNA genome, in the presence of packaging components, into a viral particle capable of infecting a target cell. Infection of the target cell includes reverse transcription and integration into the target cell genome. The RRV carries non-viral coding sequences which are to be delivered by the vector to the target cell. An RRV is incapable of independent replication to produce infectious retroviral particles within the final target cell. Usually the RRV lacks a functional gag-pol and/or env gene and/or other genes essential for replication. The vector of the present invention may be configured as a split-intron vector. A split intron vector is described in PCT patent application WO 99/15683.

A detailed list of retroviruses may be found in Coffin et al., (“Retroviruses” 1997 Cold Spring Harbour Laboratory Press Eds: J M Coffin, S M Hughes, H E Varmus pp 758-763).

Lentiviral Vectors

Lentiviruses can be divided into primate and non-primate groups. Examples of primate lentiviruses include but are not limited to: the human immunodeficiency virus (HIV), the causative agent of human auto-immunodeficiency syndrome (AIDS), and the simian immunodeficiency virus (SIV). The non-primate lentiviral group includes the prototype “slow virus” visna/maedi virus (VMV), as well as the related caprine arthritis-encephalitis virus (CAEV), equine infectious anaemia virus (EIAV) and the more recently described feline immunodeficiency virus (FIV) and bovine immunodeficiency virus (BIV).

A distinction between the lentivirus family and other types of retroviruses is that lentiviruses have the capability to infect both dividing and non-dividing cells (Lewis et al., 1992, EMBO. J 11: 3053-3058; Lewis and Emerman, 1994, J. Virol. 68: 510-516). In contrast, other retroviruses—such as MLV—are unable to infect non-dividing cells such as those that make up, for example, muscle, brain, lung and liver tissue. As lentiviruses are able to transduce terminally differentiated/primary cells, the use of a lentiviral screening strategy allows library selection in a primary target host cell.

Preferably a lentiviral screening strategy is used to identify a DAM using a eukaryotic screening system.

Preferably an EIAV/lentivirus based system is used to identify a DAM using a eukaryotic screening system.

Adenoviruses

In one embodiment of the present invention, the features of adenoviruses may be combined with the genetic stability of retroviruses/lentiviruses which can be used to transduce target cells to become transient retroviral producer cells capable of stably infect neighbouring cells. In one embodiment, such retroviral producer cells which are engineered to express an identified DAM using the method of the present invention can be implanted in organisms such as animals or humans for use in the treatment of disease such as cancer.

Pox Viral Vectors

Pox viral vectors may be used in accordance with the present invention, as large fragments of DNA are easily cloned into its genome and recombinant attenuated vaccinia variants have been described (Meyer, et al., 1991, J. Gen. Virol. 72: 1031-1038, Smith and Moss, 1983, Gene, 25:21-28).

Examples of pox viral vectors include but are not limited to leporipoxvirus: Upton, et al J. Virology 60: 920 (1986) (shope fibroma virus); capripoxvirus: Gershon, et al J. Gen. Virol. 70: 525 (1989) (Kenya sheep-1); orthopoxvirus: Weir, et al J. Virol 46: 530 (1983) (vaccinia); Esposito, et al Virology 135:561 (1984) (monkeypox and variola virus); Hruby, et al PNAS, 80:3411 (1983) (vaccinia); Kilpatrick, et al Virology 143: 399 (1985) (Yaba monkey tumour virus); avipoxvirus: Binns, et al J. Gen. Virol 69: 1275 (1988) (fowlpox); Boyle, et al Virology 156:355 (1987) (fowlpox); Schnitzlein, et al J. Virological Method, 20: 341 (1988) (fowlpox, quailpox); entomopox (Lytvyn, et al J. Gen. Virol 73: 3235-3240 (1992).

Poxvirus vectors are used extensively as expression vehicles for DAM expression in eukaryotic cells. Their ease of cloning and propagation in a variety of host cells has led, in particular, to the widespread use of poxvirus vectors for expression of foreign protein and as delivery vehicles for vaccine antigens. (Moss, B. 1991, Science 252: 1662-7).

Preferred vectors for use in accordance with the present invention are recombinant pox viral vectors such as fowl pox virus (FPV), entomopox virus, vaccinia virus such as NYVAC, canarypox virus, MVA or other non-replicating viral vector systems such as those described for example in WO 95/30018. Pox virus vectors have also been described where at least one immune evasion gene has been deleted (see WO 00/29428).

In one preferred embodiment, the pox virus vector is an entomopox virus vector.

Vaccinia Viral Vectors

Preferaly the pox viral vector is a vaccinia viral vector.

Preferably, the vector is a vaccinia virus vector such as MVA or NYVAC. Most preferred is the vaccinia strain modified virus ankara (MVA) or a strain derived therefrom. Alternatives to vaccinia vectors include avipox vectors such as fowlpox or canarypox known as ALVAC and strains derived therefrom which can infect and express recombinant proteins in human cells but are unable to replicate.

Construction of a Pox Viral Vector Library

Typically, the foreign DNA is introduced into the poxvirus genome by homologous recombination. The target DAM coding sequences are cloned behind a vaccinia promoter flanked by sequences homologous to a non-essential region in the poxvirus and the plasmid intermediate is recombined into the viral genome by homologous recombination. This methodology works efficiently for relatively small inserts tolerated by prokaryotic hosts.

The method is less viable in cases requiring large inserts as the frequency of homologous recombination is low and decreases with increasing insert size; in cases requiring construction of labor intensive plasmid intermediates such as in expression library production; and, in cases where the propagation of DNA is not tolerated in bacteria.

Alternative methods using direct ligation vectors have been developed to efficiently construct chimeric genomes in situations not readily amenable for homologous recombination (Merchlinsky, M. et al., 1992, Virology 190: 522-526; Scheiflinger, F. et al., 1992, Proc. Natl. Acad. Sci. USA. 89: 9977-9981). These direct ligation protocols have obviated the need for homologous recombination to generate poxvirus chimeric genomes. In such protocols, the DNA from the genome is digested, ligated to insert DNA in vitro, and transfected into cells infected with a helper virus (Merchlinsky, M. et al., 1992, Virology 190: 522-526, Scheiflinger, F. et al., 1992, Proc. Natl. Acad. Sci. USA 89: 977-9981). In one protocol, the genome is digested at the unique NotI site and a DNA insert containing elements for selection or detection of the chimeric genomes is ligated to the genomic arms (Scheiflinger, F. et al., 1992, Proc. Natl. Acad. Sci. USA. 89: 9977-9981). This direct ligation method is described for the insertion of foreign DNA into the vaccinia virus genome (Pfleiderer et al., 1995, J. General Virology 76: 2957-2962). Alternatively, the vaccinia WR genome is modified by removing the NotI site in the HindIII F fragment and reintroducing a NotI site proximal to the thymidine kinase gene such that insertion of a sequence at this locus disrupts the thymidine kinase gene, allowing isolation of chimeric genomes via use of drug selection (Merchlinsky, M. et al., 1992, Virology 190: 522-526).

The direct ligation vector, vNotI/tk allows one to efficiently clone and propagate DNA inserts at least 26 kilobase pairs in length (Merchlinsky, M. et al., 1992, Virology, 190: 522-526). Although, large DNA fragments are efficiently cloned into the genome, proteins encoded by the DNA insert will only be expressed at the low level corresponding to the thymidine kinase gene, a relatively weakly expressed early class gene in vaccinia. In addition, the DNA is inserted in both orientations at the NotI site.

Improved and modified vaccinia virus vectors for efficient construction of such DNA libraries can be prepared using a “trimolecular recombination” approach to improve screening efficiency.

In one embodiment of the invention, a representative DNA library is constructed in vaccinia virus. Preferably, a tri-molecular recombination method employing modified vaccinia virus vectors and related transfer plasmids is used to construct the representative DNA library in vaccinia virus. This method generates close to 100% recombinant vaccinia virus (see Section 6, Section 6.2 and 6.3 of WO 00/28016).

Tri-Molecular Recombination

The above-described tri-molecular recombination strategy yields close to 100% viral recombinants. This is a highly significant improvement over current methods for generating viral recombinants by transfection of a plasmid transfer vector into vaccinia virus infected cells. This latter procedure yields viral recombinants at a frequency of the order of only 0.1%. The high yield of viral recombinants in tri-molecular recombination makes it possible to efficiently construct genomic or cDNA libraries in a vaccinia virus derived vector. A titer of 6×10 recombinant virus can be obtained following transfection with a mix of 20 micrograms of Not I and Apa I digested vaccinia vector arms together with an equimolar concentration of tumor cell cDNA. This technological advance creates the possibility of new and efficient screening and selection strategies for isolation of specific genomic and cDNA clones.

The tri-molecular recombination method as herein disclosed may be used with other viruses such as mammalian viruses including vaccinia and herpes viruses. Typically, two viral arms which have no homology are produced. The only way that the viral arms can be linked is by bridging through homologous sequences that flank the insert in a transfer vector such as a plasmid. When the two viral arms and the transfer vector are present in the sarne cell the only infectious virus produced is recombinant for a DNA insert in the transfer vector.

Libraries constructed in vaccinia and other mammalian viruses by the tri-molecular recombination method of the present invention may be used in identifying target DAMs in the screening system of the present invention.

Hybrid Viral Vectors

In a further embodiment, the present invention provides a hybrid viral vector system for in vivo delivery of a nucleotide sequence encoding an DAM identified by the method of the present invention, which system comprises one or more primary viral vectors which encode a secondary viral vector, the primary vector or vectors capable of infecting a first target cell and of expressing therein the secondary viral vector, which secondary vector is capable of transducing a secondary target cell.

Minimal Viral Genome

Preferably the viral vector of the present invention has a minimal viral genome.

As used herein, the term “minimal viral genome” means that the viral vector has been manipulated so as to remove the non-essential elements and to retain the essential elements in order to provide the required functionality to infect, transduce and deliver a nucleotide sequence of interest to a target host cell.

Preferably the viral vector with the minimal viral genome is a pox viral vector.

Preferably the viral vector with the minimal viral genome is a lentiviral vector.

Host Cells

Host cells may be used to express the target DAM of the present invention.

The term “host cell” includes any cell derivable from a suitable organism which a vector is capable of transfecting or transducing.

Organism

The term “organism” includes any suitable organism. In a preferred embodiment, the organism is a mammal. In a highly preferred embodiment, the organism is a human.

Eukaryotic

The target DAM of the present invention is identified by expression of a cDNA library from eukaryotic host cells. As used herein, the term “eukaryotic cell” means a cell with a cell nucleus which is bounded by a nuclear membrance and contains true chromosomes.

It is characteristic of all multicellular and unicellular organisms except bacteria, actinomycetes and cyanobacteria. Examples of eukaryotic host cells include but are not limited to yeast, insect or mammalian cells, in particular mammalian cells. Suitable host cells include, yeast, mammalian cell lines and other eukaryotic cell lines, for example insect Sf9 cells.

Other examples of host cells can include but are not limited to cells capable of expressing the target DAM of the present invention. Examples of such cells include but are not limited to macrophages, endothelial cells or combinations thereof. Further examples include respiratory airway epithelial cells, hepatocytes, muscle cells, cardiac myocytes, synoviocytes, primary mammary epithelial cess and post-mitotically terminally differentiated non-replicating cells such as macrophages and/or neurons.

Post Translational Modification

A host cell strain may be chosen which modulates the expression of the inserted cDNA library nucleotide sequences, or modifies and processes the expressed DAM in the specific fashion desired. Such modifications (e.g., glycosylation) and processing (e.g. cleavage) of protein products may be important for the function of the protein. Different host cells have characteristic and specific mechanisms for the posttranslational processing and modification of proteins and gene products. Appropriate cell lines or host systems can be chosen to ensure the correct modification of the foreign protein expressed. To this end, eucaryotic host cells which possess the cellular machinery for proper processing of the primary transcript, glycosylation, acetylation and phosphorylation of the gene product may be used. Such mammalian host cells include but are not limited to CHO, VERO, BHK, HeLa, COS, MDCK, 293, 3T3 and WI38 cell lines.

In a preferred embodiment, the cell is a mammalian cell.

In a highly preferred embodiment, the cell is a human cell.

The present invention provides a method comprising transforming a host and/or target cell with an expression library of the present invention.

The term “transformed cell” means a host cell having a modified genetic structure. With the present invention, a cell has a modified genetic structure when a vector according to the present invention has been introduced into the cell.

Host cells and/or a target cells may be cultured under suitable conditions which allow expression of the target DAM of the invention.

The present invention also provides a method comprising culturing a transformed host cell—which cell has been transformed with a vector according to the present invention under conditions suitable for the expression of the DAM of the present invention.

The expressed target DAM of the present invention is contacted with a sample comprising a binding partner.

Sample

As used herein, the term “sample” may include but is not limited to a sample obtained from a subject or a sample obtained from an animal or a sample of tissue or a sample of body fluid.

The term “tissue” is used herein to refer to any biological matter made up of one cell, multiple cells, an agglomeration of cells, or an entire organ. The term tissue also encompasses a cell or cells which can be either normal or abnormal (i.e. a tumour). A “body fluid” may be any liquid substance extracted, excreted, or secreted from an organism or a tissue of an organism. The body fluid need not necessarily contain cells. Body fluids of relevance to the present invention include, but are not limited to, whole blood, serum, plasma, urine, cerebral spinal fluid, tears, and amniotic fluid.

Detection of expression of the target DAM of the present invention may be achieved, for instance, by the application of a binding partner capable of specifically reacting with the DAM expression product.

Agent

As used herein, the term “agent” means any entity that is capable of detecting a host cell expressing a target DAM.

Binding Partner (BP)

Preferably, the binding partner (BP) comprises one or more binding domains capable of binding to one or more of the host cell expressing a DAM. Thus the BP is directed to a particular cell by its affinity for a target DAM.

The one or more binding domains of the BP may consist of, for example, a natural ligand for a DAM, which natural ligand may be an adhesion molecule or a growth-factor receptor ligand (eg epidermal growth factor), or a fragment of a natural ligand which retains binding affinity for the DAM.

Alternatively, the binding domains may be derived from heavy and light chain sequences from an immunoglobulin (Ig) variable region. Such a variable region may be derived from a natural human antibody or an antibody from another species such as a rodent antibody. Alternatively the variable region may be derived from an engineered antibody such as a humanised antibody or from a phage display library from an immunised or a non-immunised animal or a mutagenised phage-display library. As a second alternative, the variable region may be derived from a single-chain variable fragment (scFv). The BP may contain other sequences to achieve multimerisation or to act as spacers between the binding domains or which result from the insertion of restriction sites in the genes encoding the BP, including Ig hinge sequences or novel spacers and engineered linker sequences.

The BP may comprise, in addition to one or more immunoglobulin variable regions, all or part of an Ig heavy chain constant region and so may comprise a natural whole Ig, an engineered Ig, an engineered Ig-like molecule, a single-chain Ig or a single-chain Ig-like molecule. Alternatively, or in addition, the BP may contain one or more domains from another protein such as a toxin.

Antibody

Preferably the binding partner is an antibody.

As used herein, an “antibody” refers to a protein consisting of one or more polypeptides substantially encoded by immunoglobulin genes or fragments of immunoglobulin genes. Antibodies may exist as intact immunoglobulins or as a number of fragments, including those well-characterized fragments produced by digestion with various peptidases. While various antibody fragments are defined in terms of the digestion of an intact antibody, one of skill will appreciate that antibody fragments may be synthesized de novo either chemically or by utilizing recombinant DNA methodology. Thus, the term antibody, as used herein also includes antibody fragments either produced by the modification of whole antibodies or synthesized de novo using recombinant DNA methodologies. Antibody fragments encompassed by the use of the term “antibodies” include, but are not limited to, Fab, Fab′, F (ab′) 2, scFv, Fv, dsFv diabody, and Fd fragments.

Antibodies specifically inimunoreactive with the target DAM of the present invention represent still another embodiment of the invention. These antibodies may be monoclonal or polyclonal. The antibodies may optionally be recombinant or purely synthetic. The antibody may be an intact antibody or fragment. The preparation of antibodies specific to the DAM of the present invention would be routine for those skilled in the art.

An antibody array may be used in the present invention. The antibodies on the array may be monoclonal or polyclonal. They may be intact antibodies or fragments of antibodies that are capable of specifically binding the polypeptides of the present invention.

Preferably the antibody array comprises at least four different antibodies, and preferably more than about 10 different antibodies. For instance, methods of assaying for expression of a target DAM, comprises first contacting a sample of body fluid or tissue obtained from the animal or an antibody array with a cDNA clone from an expression library of the present invention. The target DAM may be contacted with tissue or fluid samples from an animal or directly with an antibody array, and binding of the DAM to the antibody on the array detected. Alternatively, the tissue or fluid sample may be purified to isolate the antibody or mRNA transcripts prior to contact with the cDNA clone.

Preferably the antibody is a polyclonal antibody.

Preferably the antibody is a monoclonal antibody.

Preferably the antibody is of high affinity and titre.

If the antibody is of low affinity and/or titre, then preferably the DAM is expressed at high levels.

Screens for the Target Dam

Detection of expression of the target DAM of the present invention may be achieved, for instance, by the application of labeled antibodies specifically immunoreactive with the DAM expression product. The antibodies may be derived from tissue or from body fluid samples removed from a human or an animal. Various forms of typical immunoassays known to those skilled in the art would be applicable here. These assays include both competitive and non-competitive assays. For instance, in one type of assay sometimes referred to as a “sandwich assay”, immobilized antibodies that specifically react with DAM are contacted with the biological tissue or fluid sarnple. The presence of the immobilized DAM-antibody complex could then be achieved by application of a second, labeled antibody immunoreactive with either the DAM or the DAM-antibody complex. A Western blot type of assay could also be used in an alternative embodiment of the present invention.

Regulation of Expression

Preferably the expression of the DAM is regulated to modify the rate of transcription or translation.

In one embodiment, the present invention also encompasses control regions/sequences associated with the DAM or encoding sequence thereof to modify the rate of transcription or translation.

Control Sequences

Control sequences operably linked to sequences encoding the DAM of the present invention include promoters/enhancers and other expression regulation signals. These control sequences may be selected to be compatible with the host cell and/or target cell in which the expression vector is designed to be used. The control sequences may be modified, for example by the addition of further transcriptional regulatory elements to make the level of transcription directed by the control sequences more responsive to transcriptional modulators.

Operably Linked

The term “operably linked” means that the components described are in a relationship permitting them to function in their intended manner. A regulatory sequence “operably linked” to a coding sequence is ligated in such a way that expression of the coding sequence is achieved under condition compatible with the control sequences.

Preferably the nucleotide sequence encoding the target DAM of the present invention is operably linked to a transcription unit.

The term “transcription unit(s)” as described herein are regions of nucleic acid containing coding sequences and the signals for achieving expression of those coding sequences independently of any other coding sequences. Thus, each transcription unit generally comprises at least a promoter, an optional enhancer and a polyadenylation signal.

Promoters

The term promoter is well-known in the art and is used in the normal sense of the art, e.g. as an RNA polymerase binding site. The term encompasses nucleic acid regions ranging in size and complexity from minimal promoters to promoters including upstream elements and enhancers.

The promoter can include features to ensure or to increase expression. For example, the features can be conserved regions such as a Pribnow Box, Kozak sequence or a TATA box. The promoter may even contain other sequences to affect (such as to maintain, enhance, decrease) the levels of expression of the nucleotide sequence of the present invention. For example, suitable other sequences include the Sh1-intron or an ADH intron. Other sequences include inducible elements—such as temperature, chemical, light or stress inducible elements. Also, suitable elements to enhance transcription or translation may be present. An example of the latter element is the TMV 5′ signal sequence (see Sleat, 1987, Gene 217: 217-225; Dawson 1993, Plant Mol. Biol. 23: 97).

The promoter is typically selected from promoters which are functional in mammalian, cells, although promoters functional in other eukaryotic cells may be used. The promoter is typically derived from promoter sequences of viral or eukaryotic genes. For example, it may be a promoter derived from the genome of a cell in which expression is to occur. With respect to eukaryotic promoters, they may be promoters that function in a ubiquitous manner (such as promoters of α-actin, β-actin, tubulin) or, alternatively, a tissue-specific manner (such as promoters of the genes for pyruvate kinase).

Preferably the promoter is a modified H5 or sE/L promoter. For more information see Carroll et al (2001).

Preferably the promoter is a early late promoter is used for maximum protein production ans which allows optimal sensitivity during antibody identification process

Preferably the promoter is designed using data in Davison & Moss (J. Mol. Biol. 1989 210: 749-769).

Hypoxic Promoters/Enhancers

The enhancer and/or promoter may be preferentially active in a hypoxic or ischaemic or low glucose environment, such that the DAM encoding nucleotide sequence(s) is preferentially expressed when the host cell is cultivated under certain conditions such as ischaemic conditions. The enhancer element or other elements conferring regulated expression may be present in multiple copies. Likewise, or in addition, the enhancer and/or promoter may be preferentially active in one or more specific host cell types—such as any one or more of macrophages, endothelial cells or combinations thereof. Further examples may include but are not limited to respiratory airway epithelial cells, hepatocytes, muscle cells, cardiac myocytes, synoviocytes, primary mammary epithelial cells and post-mitotically terminally differentiated non-replicating cells such as macrophages and/or neurons.

Tissue-Specific Promoters

The promoters of the present invention may be tissue-specific promoters. Examples of suitable tissue restricted promoters/enhancers are those which are highly active in tumour cells such as a promoter/enhancer from a MUC1 gene, a CEA gene or a 5T4 antigen gene. Examples of temporally restricted promoters/enhancers are those which are responsive to ischaemia and/or hypoxia, such as hypoxia response elements or the promoter/enhancer of a grp78 or a grp94 gene. The alpha fetoprotein (AFP) promoter is also a tumour-specific promoter. One preferred promoter-enhancer combination is a human cytomegalovirus (hCMV) major immediate early (MIE) promoter/enhancer combination.

Preferably the promoters of the present invention are tissue specific.

The term “tissue specific” means a promoter which is not restricted in activity to a single tissue type but which nevertheless shows selectivity in that they may be active in one group of tissues and less active or silent in another group. A desirable characteristic of the promoters of the present invention is that they posess a relatively low activity in the absence of activated hypoxia-regulated enhancer elements. One means of achieving this is to use “silencer” elements which suppress the activity of a selected promoter in the absence of hypoxia.

The term “hypoxia” means a condition under which a particular organ or tissue receives an inadequate supply of oxygen.

The level of expression of a DAM encoding nucleotide sequence(s) under the control of a particular promoter may be modulated by manipulating the promoter region. For example, different domains within a promoter region may possess different gene regulatory activities. The roles of these different regions are typically assessed using vector constructs having different variants of the promoter with specific regions deleted (that is, deletion analysis). This approach may be used to identify, for example, the smallest region capable of conferring tissue specificity or the smallest region conferring hypoxia sensitivity.

A number of tissue specific promoters, described above, may be particularly advantageous in practising the present invention. In most instances, these promoters may be isolated as convenient restriction digestion fragments suitable for cloning in a selected vector. Alternatively, promoter fragments may be isolated using the polymerase chain reaction. Cloning of the amplified fragments may be facilitated by incorporating restriction sites at the 5′ end of the primers.

Inducible Promoters

The promoters of the present invention may also be promoters that respond to specific stimuli, for example promoters that bind steroid hormone receptors.

Viral promoters may also be used, for example the Moloney murine leukaemia virus long terminal repeat (MMLV LTR) promoter, the rous sarcoma virus (RSV) LTR promoter or the human cytomegalovirus (CMV) IE promoter.

It may also be advantageous for the promoters to be inducible so that the levels of expression of the target DAM can be regulated during the lifetime of the cell. Inducible means that the levels of expression obtained using the promoter can be regulated.

Enhancer

In addition, any of these promoters may be modified by the addition of further regulatory sequences, for example enhancer sequences. Chimeric promoters may also be used comprising sequence elements from two or more different promoters described above.

The term “enhancer” includes a DNA sequence which binds to other protein components of the transcription initiation complex and thus facilitates the initiation of transcription directed by its associated promoter.

Combination of the Identified DAM with POIs/NOIs

The identified DAM of the present invention may be used in combination with a protein of interest (POI) or a nucleotide sequence of interest (NOI) encoding same. In one embodiment, the identified DAM of the present invention or nucleotide sequence encoding same may be used in combination with a POI, such as a pro-drug activating enzyme either directly or by vector delivery to, for example, a target cell or target tissue. Instead of or as well as being selectively expressed in target tissues, the identified DAM of the present invention or nucleotide sequence encoding same may be used in combination with another POI such as a pro-drug activation enzyme or enzymes or with a nucleotide sequences of interest (NOI) or NOIs which encode a pro-drug activation enzyme or enzymes. These pro-drug activation enzyme or enzymes may have no significant effect or no deleterious effect until the individual is treated with one or more pro-drugs upon which the appropriate pro-drug enzyme or enzymes act. In the presence of the active POI or NOI encoding same, treatment of an individual with the appropriate pro-drug may lead to enhanced reduction in the disease condition such as a reduction in tumour growth or survival.

POIs AND NOIs

Other suitable proteins of interest (POIs) or NOIs encoding same for use in the present invention with the identified DAM include those that are of therapeutic and/or diagnostic application such as, but are not limited to: sequences encoding cytokines, chemokines, hormones, antibodies, engineered immunoglobulin-like molecules, a single chain antibody, fusion proteins, enzymes, immune co-stimulatory molecules, immunomodulatory molecules, anti-sense RNA, a transdominant negative mutant of a target protein, a toxin, a conditional toxin, an antigen, a tumour suppressor protein and growth factors, membrane proteins, vasoactive proteins and peptides, anti-viral proteins and ribozymes, and derivatives therof (such as with an associated reporter group). When included, the POIs or NOIs encoding same may be typically operatively linked to a suitable promoter, which may be a promoter driving expression of a ribozyme(s), or a different promoter or promoters, such as in one or more specific cell types.

Suitable POIs or NOIs encoding same for use in the present invention in combination with the identified DAM in the treatment or prophylaxis of cancer include proteins which: destroy the target cell (for example a ribosomal toxin), act as: tumour suppressors (such as wild-type p53); activators of anti-tumour immune mechanisms (such as cytokines, co-stimulatory molecules and immunoglobulins); inhibitors of angiogenesis; or which provide enhanced drug sensitivity (such as pro-drug activation enzymes); indirectly stimulate destruction of target cell by natural effector cells (for example, strong antigen to stimulate the immune system or convert a precursor substance to a toxic substance which destroys the target cell (for example a prodrug activating enzyme). Encoded proteins could also destroy bystander tumour cells (for example with secreted anti-tumour antibody-ribosomal toxin fusion protein), indirectly stimulate destruction of bystander tumour cells (for example cytokines to stimulate the immune system or procoagulant proteins causing local vascular occlusion) or convert a precursor substance to a toxic substance which destroys bystander tumour cells (eg an enzyme which activates a prodrug to a diffusible drug).

Also, the delivery of NOI(s) encoding antisense transcripts or ribozymes which interfere with expression of cellular genes for tumour persistence (for example against aberrant myc transcripts in Burkitts lymphoma or against bcr-abl transcripts in chronic myeloid leukemia. The use of combinations of such POIs and/or NOIs encoding same is also envisaged.

Examples of hypoxia regulatable therapeutic NOIs can be found in PCT/GB95/00322 (WO-A-9521927).

Vaccines

Since the identified DAM of the present invention can be produced in large amounts, the antigen thus produced and purified has use in vaccine preparations. The DAM may be formulated into a subunit vaccine preparation, or may be engineered into viral vectors and formulated into vaccine preparations. Alternatively, the DNA encoding the identified DAM may be administered directly as a vaccine formulation. The “naked” plasmid DNA once administered to a subject invades cells, is expressed on the surface of the invaded cell and elicits a cellular immune response, so that T lymphocytes will attack cells displaying the identified DAM. The identified DAM also has utility in diagnostics, for example, to detect or measure in a sample of body fluid from a subject the presence of tumors and thus to diagnose cancer and tumors and/or to monitor the cellular immune response of the subject subsequent to vaccination.

The recombinant viruses of the invention can be used to treat tumor-bearing mammals, including humans, to generate an immune response against the tumor cells. The generation of an adequate and appropriate immune response leads to tumor regression in vivo. Such “vaccines” can be used either alone or in combination with other therapeutic regimens, including but not limited to chemotherapy, radiation therapy, surgery, bone marrow transplantation, etc. for the treatment of tumors. For example, surgical or radiation techniques could be used to debulk the tumor mass, after which, the vaccine formulations of the invention can be administered to ensure the regression and prevent the progression of remaining tumor masses or micrometastases in the body. Alternatively, administration of the “vaccine” can precede such surgical, radiation or chemotherapeutic treatment.

Alternatively, the recombinant viruses of the invention can be used to immunize or “vaccinate” tumor-free subjects to prevent tumor formation. With the advent of genetic testing, it is now possible to predict a subject's predisposition for cancers. Such subjects, therefore, may,: for example, be immunized using a recombinant vaccinia virus expressing an appropriate DAM, such as a tumor-associated antigen (TAA). The immunopotency of the DAM vaccine formulations antigen can be determined by monitoring the immune response in test animals following immunization or by use of any immunoassay known in the art. Generation of a cell-mediated immune response may be taken as an indication of an immune response. Test animals may include mice, hamsters, dogs, cats, monkeys, rabbits, chimpanzees, etc., and eventually human subjects.

Suitable preparations of such vaccines include injectables, either as liquid solutions or suspensions; solid forms suitable for solution in, suspension in, liquid prior to injection, may also be prepared. The preparation may also be emulsified, or the polypeptides encapsulated in liposomes. The active immunogenic ingredients are often mixed with excipients which are pharmaceutically acceptable and compatible with the active ingredient. Suitable excipients are, for example, water, saline, dextrose, glycerol, ethanol, or the like and combinations thereof.

In addition, if desired, the vaccine preparation may also include minor amounts of auxiliary substances such as wetting or emulsifying agents, pH buffering agents, and/or adjuvants which enhance the effectiveness of the vaccine.

Examples of adjuvants which may be effective, include, but are not limited to: aluminum hydroxide, Nacetyl-muramyl-L-threonyl-D-isoglutamine (thr-MDP), Nacetyl-nor-muramyl-L-alanyl-D-isoglutamine, acetylmuramyl-L-alanyl-D-isoglutaminyl-L-alanine-2-(1′-2′dipalmitoyl-sn-glycero-3-hydroxyphosphoryloxy)-ethylamine, M-CSF, QS-21 (investigational drug, Progenics harmaceuticals, Inc.), DETOX (investigational drug, Ribi Pharmaceuticals), and BCG.

The effectiveness of an adjuvant may be determined by measuring the induction of the cellular immune response directed against the identified DAM.

The vaccines of the invention may be multivalent or univalent. Multivalent vaccines are made from recombinant viruses that direct the expression of more than one antigen.

The composition, if desired, can also contain minor amounts of wetting or emulsifying agents, or pH buffering agents. The composition can be a liquid solution, suspension, emulsion, tablet, pill, capsule, sustained release formulation, or powder. Oral formulation can include standard carriers such as pharmaceutical grades of mannitol, lactose, starch, magnesium stearate, sodium saccharine, cellulose, magnesium carbonate, etc.

Generally, the ingredients are supplied either separately or mixed together in unit dosage form, for example, as a dry lyophilized powder or water free concentrate in a hermetically sealed container such as an ampoule or sachette indicating the quantity of active agent. Where the composition is administered by injection, an ampoule of sterile diluent can be provided so that the ingredients may be mixed prior to administration.

In a specific embodiment, a lyophilized epitope of the invention is provided in a first container; a second container comprises diluent consisting of an aqueous solution of 50 t glycerin, 0.25 t phenol, and an antiseptic (e.g., 0.005-i5 brilliant green).

Use of purified DAMs as vaccine preparations can be carried out by standard methods. For example, the purified protein (s) should be adjusted to an appropriate concentration, formulated with any suitable vaccine adjuvant and packaged for use. Suitable adjuvants may include, but are not limited to: mineral gels, e.g., aluminum hydroxide; surface active substances such as lysolecithin, pluronic polyols; polyanions; peptides; oil emulsions; alum, and MDP. The immunogen may also be incorporated into liposomes, or conjugated to polysaccharides and/or other polymers for use in a vaccine formulation. In instances where the recombinant antigen is a hapten, i.e., a molecule that is antigenic in that it can react selectively with cognate antibodies, but not immunogenic in that it cannot elicit an immune response, the hapten may be covalently bound to a carrier or immunogenic molecule; for instance, a large protein such as serum albumin will confer immunogenicity to the hapten coupled to it. The hapten-carrier may be formulated for use as a vaccine.

Many methods may be used to introduce the vaccine formulations described above into a patient. These include, but are not limited to, oral, intradermal, intramuscular, intraperitoneal, intravenous, subcutaneous, intranasal, transdermal, epidural, pulmonary, gastric, intestinal, rectal, vaginal, or urethral routes. When the method of treatment uses a live recombinant vaccinia vaccine formulation of the invention, it may be preferable to introduce the formulation via the natural route of infection of the vaccinia virus, i.e., through a mucosal membrane or surface, such as an oral, nasal, gastric, ntestinal, rectal, vaginal or urethral route. To induce a TL response, the mucosal route of administration may be through an oral or nasal membrane. Alternatively, an intramuscular or intraperitoneal route of administration may be used. Preferably, a dose of 10″-10′PFU (plaque forming units) of cold adapted recombinant vaccinia virus is given to a human patient.

The precise dose of vaccine preparation to be employed in the formulation will also depend on the route of administration, and the nature of the patient, and should be decided according to the judgment of the practitioner and each patient's circumstances according to standard clinical techniques. An effective immunizing amount is that amount sufficient to produce an immune response to the antigen in the host to which the vaccine preparation is administered.

Where subsequent or booster doses are required, a modified vaccinia virus such as MVA can be elected as the parental virus used to generate the recombinant.

Alternatively, another virus, e.g., adenovirus, canary pox virus, or a subunit preparation can be used to boost.

Immunization and/or cancer immunotherapy may be accomplished using a combined immunization egimen, e.g., immunization with a recombinant vaccinia viral vaccine of the invention and a boost f a recombinant vaccinia viral vaccine. In such an embodiment, a strong secondary CD8 T cell response is induced after priming and boosting with different viruses expressing the same epitope for such methods of immunization and boosting, see, e.g., Murata et al., Cellular Immunol. 173: 6-107). For example, a patient is first primed with a vaccine formulation of the invention comprising recombinant vaccinia virus expressing an epitope, e.g., a selected tumor-associated antigen or ragment thereof. The patient is then boosted, e. Q., 21 days later, with a vaccine formulation comprising a recombinant virus other than vaccinia expressing the same epitope. Such priming followed by boosting induces a strong secondary CD8T cell response. Such a priming and boosting immunization regimen is preferably used to treat a patient with a tumor, metastasis or neoplastic growth expressing the selected tumor-associated antigen.

In yet another embodiment, the recombinant vaccinia viruses can be used as a booster immunization subsequent to a primary immunization with inactivated tumor cells, a subunit vaccine containing the tumor-associated antigen or its epitope, or another recombinant viral vaccine, e.g., adenovirus, canary pox virus, or MVA.

In an alternate embodiment, recombinant vaccinia virus encoding a particular tumor-associated antigen, epitope or fragment thereof may be used in adoptive immunotherapeutic methods for the activation of T lymphocytes that are histocompatible with the patient and specific for the tumor-associated antigen (for methods of adoptive immunotherapy, see, e.g., Rosenberg, U.S. Pat. No. 4,690,915, issued Sep. 1, 1987; Zarling, et al., U.S. Pat. No. 5,081,029, issued Jan. 14, 1992). Such T lymphocytes may be isolated from the patient or a histocompatible donor. The T lymphocytes are activated in vitro by exposure to the recombinant vaccinia virus of the invention. Activated T lymphocytes are expanded and inoculated into the patient in order to transfer T cell immunity directed against the tumor-associated antigen epitope.

The invention also provides a pharmaceutical pack or kit comprising one or more containers comprising one or more of the ingredients of the vaccine formulations of the invention. Associated with such container (s) can be a notice in the form prescribed by a governmental agency regulating the manufacture, use or sale of pharmaceuticals or biological products, which notice reflects approval by the agency of manufacture, use or sale for human administration.

Dosage

The dosage of the identified DAM of the present invention will depend on the disease state or condition being treated and other clinical factors such as weight and condition of the human or animal and the route of administration of the compound. Depending upon the half-life of the DAM in the particular animal or human, the DAM can be administered between several times per day to once a week. It is to be understood that the present invention has application for both human and veterinary use. The methods of the present invention contemplate single as well as multiple administrations, given either simultaneously or over an extended period of time.

Formulations

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampoules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the kind previously described.

The identified DAM of the present invention may be effective in preventing and/or treating diseases such as cancer related diseases. The present invention includes the method of treating diseases such as cancer related disease with an effective amount of an identified DAM of the present invention. The identified DAM of the present invention can be provided as a synthetic peptide or an isolated and substantially purified proteins or protein fragments or a combination thereof in pharmaceutically acceptable compositions using formulation methods known to those of ordinary skill in the art. These compositions can be administered by standard routes. These include but are not limited to: oral, rectal, ophthalmic (including intravitreal or intracameral), nasal, topical (including buccal and sublingual), intrauterine, vaginal or parenteral (including subcutaneous, intraperitoneal, intramuscular, intravenous, intradermal, intracranial, intratracheal, and epidural) transdermal, intraperitoneal, intracranial, intracerebroventricular, intracerebral, intravaginal, intrauterine, or parenteral (e.g., intravenous, intraspinal, subcutaneous or intramuscular) routes.

The DAM formulations may conveniently be presented in unit dosage form and may be prepared by conventional pharmaceutical techniques. Such techniques include the step of bringing into association the active ingredient and the pharmaceutical carrier(s) or excipient(s). In general, the formulations are prepared by uniformly and intimately bringing into association the active ingredient with liquid carriers or finely divided solid carriers or both, and then, if necessary, shaping the product.

In addition, the identified DAM of the present invention may be incorporated into biodegradable polymers allowing for sustained release of the compound, the polymers being implanted in the vicinity of where drug delivery is desired, for example, at the site of a tumor or implanted so that the DAM is slowly released systemically. The biodegradable polymers and their use are described, for example, in detail in Brem et al (J. Neurosurg 1991, 74: 441-446). Osmotic minipumps may also be used to provide controlled delivery of high concentrations of DAM through cannulae to the site of interest, such as directly into a metastatic growth or into the vascular supply to that tumor.

The identified DAM of the present invention may be linked to cytotoxic agents which are infused in a manner designed to maximize delivery to the desired location. For example, ricin-linked high affinity DAMs are delivered through a cannula into vessels supplying the target site or directly into the target. Such agents are also delivered in a controlled manner through osmotic pumps coupled to infusion cannulae.

Preferred unit dosage formulations are those containing a daily dose or unit, daily sub-dose, as herein above recited, or an appropriate fraction thereof, of the administered ingredient. It should be understood that in addition to the ingredients, particularly mentioned above, the formulations of the present invention may include other agents conventional in the art having regard to the type of formulation in question.

Pharmaceutical Compositions

In one aspect, the present invention provides a pharmaceutical composition, which comprises an identified DAM according to the present invention and optionally a pharmaceutically acceptable carrier, diluent or excipient (including combinations thereof).

The pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilizers, dyes and even flavouring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition of the present invention may be formulated to be delivered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be delivered by both routes.

Where the pharmaceutical composition is to be delivered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose or chalk, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

Administration

Typically, a physician will determine the actual dosage which will be most suitable for an individual subject and it will vary with the age, weight and response of the particular patient and severity of the condition. The dosages below are exemplary of the average case. There can, of course, be individual instances where higher or lower dosage ranges are merited.

The compositions (or component parts thereof) of the present invention may be administered orally. In addition or in the alternative the compositions (or component parts thereof) of the present invention may be administered by direct injection. In addition or in the alternative the compositions (or component parts thereof) of the present invention may be administered topically. In addition or in the alternative the compositions (or component parts thereof) of the present invention may be administered by inhalation. In addition or in the alternative the compositions (or component parts thereof) of the present invention may also be administered by one or more of: parenteral, mucosal, intramuscular, intravenous, subcutaneous, intraocular or transdermal administration means, and are formulated for such administration.

By way of further example, the pharmaceutical composition of the present invention may be administered in accordance with a regimen of 1 to 10 times per day, such as once or twice per day. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The term “administered” also includes but is not limited to delivery by a mucosal route, for example, as a nasal spray or aerosol for inhalation or as an ingestable solution; a parenteral route where delivery is by an injectable form, such as, for example, an intravenous, intramuscular or subcutaneous route.

Hence, the pharmaceutical composition of the present invention may be administered by one or more of the following routes: oral administration, injection (such as direct injection), topical, inhalation, parenteral administration, mucosal administration, intramuscular administration, intravenous administration, subcutaneous administration, intraocular administration or transdermal administration.

Diseases

Pharmaceutical compositions comprising an effective amount of an identified DAM and/or an NOI encoding same may be used in the treatment of disorders such as those listed in WO-A-98/09985. For ease of reference, part of that list is now provided: macrophage inhibitory and/or T cell inhibitory activity and thus, anti-inflammatory activity; anti-immune activity, i.e. inhibitory effects against a cellular and/or humoral immune response, including a response not associated with inflammation; diseases associated with viruses and/or other intracellular pathogens; inhibit the ability of macrophages and T cells to adhere to extracellular matrix components and fibronectin, as well as up-regulated fas receptor expression in T cells; inhibit unwanted immune reaction and inflammation including arthritis, including rheumatoid arthritis, inflammation associated with hypersensitivity, allergic reactions, asthma, systemic lupus erythematosus, collagen diseases and other autoimmune diseases, inflammation associated with atherosclerosis, arteriosclerosis, atherosclerotic heart disease, reperfusion injury, cardiac arrest, myocardial infarction, vascular inflammatory disorders, respiratory distress syndrome or other cardiopulmonary diseases, inflammation associated with peptic ulcer, ulcerative colitis and other diseases of the gastrointestinal tract, hepatic fibrosis, liver cirrhosis or other hepatic diseases, thyroiditis or other glandular diseases, glomerulonephritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune-related testicular diseases, placental dysfunction, placental insufficiency, habitual abortion, eclampsia, pre-eclampsia and other immune and/or inflammatory-related gynaecological diseases, posterior uveitis, intermediate uveitis, anterior uveitis, conjunctivitis, chorioretinitis, uveoretinitis, optic, neuritis, intraocular inflammation, e.g. retinitis or cystoid macular oedema, sympathetic ophthalmia, scleritis, retinitis pigmentosa, immune and inflammatory components of degenerative fondus disease, inflammatory components of ocular trauma, ocular inflammation caused by infection, proliferative vitreo-retinopathies, acute ischaemic optic neuropathy, excessive scarring, e.g. following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of stokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, myasthenia gravis, pseudo-tumour cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery, bone marrow transplantation or other transplantation complications and/or side effects, inflammatory and/or immune complications and side effects of gene therapy, e.g. due to infection with a viral carrier, or inflammation associated with AIDS, to suppress or inhibit a humoral and/or cellular immune response, to treat or ameliorate monocyte or leukocyte proliferative diseases, e.g. leukaemia, by reducing the amount of monocytes or lymphocytes, for the prevention and/or treatment of graft rejection in cases of transplantation of natural or artificial cells, tissue and organs such as cornea, bone marrow, organs, lenses, pacemakers, natural or artificial skin tissue. Specific cancer related disorders include but not limited to: solid tumours; blood born tumours such as leukemias; tumor metastasis; benign tumours, for example hemangiomas, acoustic neuromas, neurofibromas, trachomas, and pyogenic granulomas; rheumatoid arthritis; psoriasis; ocular angiogenic diseases, for example, diabetic retinopathy, retinopathy of prematurity, macular degeneration, corneal graft rejection, neovascular glaucoma, retrolental fibroplasia, rubeosis; Osler-Webber Syndrome; myocardial angiogenesis; plaque neovascularization; telangiectasia; hemophiliac joints; angiofibroma; wound granulation; coromay collaterals; cerebral collaterals; arteriovenous malformations; ischeniic limb angiogenesis; neovascular glaucoma; retrolental fibroplasia; diabetic neovascularization; heliobacter related diseases, fractures, vasculogenesis, hematopoiesis, ovulation, menstruation and placentation.

### EXAMPLES

The invention will now be further described by way of examples and results.

### Example 1

Preparation of a cDNA Library

A cDNA library is prepared from target cell which expresses a DAM recognised by a binding partner.

Protocols for the generation of cDNA libraries through reverse transcription of mRNA sequences are well known in the art and kits for doing so are commercially available (from Gibco BRL, for instance). In a preferred embodiment of the method, the cDNAs are synthesized by using a mixture of oligo-dT primers containing equal proportions of oligomers having a G, A, or C residue at the 3′-end (“indexed” or “registered” primers). This approach ensures that a given primer will hybridize at the start of a polyA tail sequence of an mRNA rather than randomly within the sequence. These oligo-dT primers also have a defined DNA sequence (20 to 24 base pairs in length) that is incorporated into each cDNA fragment. This tag permits the use of two PCR primers to specifically amplify the 3′-end of each cDNA. The two cDNA libraries are digested separately with restriction enzymes and then linker sequences are ligated to the ends of digested cDNA fragments. Restriction digests and ligation of linkers may be performed in any manner known to those skilled in the art. Some examples of such methods may be found in Sambrook et al. (1989) Molecular Cloning: A Laboratory Manual, 2nd. ed, Cold Spring Harbor Laboratory Press, herein incorporated by reference.

The cDNA library from one of two cell populations may be amplified with tagged oligonucleotide primers by means of the polymerase chain reaction (PCR). In a preferred embodiment, the “tag” on the oligonucleotide primers is biotin. However, any chemical or biological moiety which provides a means of selection or isolation of the tagged entity (by affinity chromatography, for instance) is suitable as a tag. In the preferred embodiment, use of biotin as a tag allows for removal of the tagged sequences on a streptavidin resin. In an alternative embodiment, however, oligonucleotides bearing a thiol group, for example, may instead be used as the tagged primer, since oligonucleotides with attached thiol groups can be retained on a variety of affinity resins, such as thiopropyl sepharose columns or mercurial resins. The cDNA library PCR-amplified with tagged primers is referred to herein as “driver” cDNA. The cDNA library from a target cell is amplified with normal, non tagged, oligonucleotide primers in a separate polymerase chain reaction. The cDNA PCR-amplified in this manner is referred to herein as “tester” cDNA. The non-tagged, amplified, tester cDNA is heated and then re-annealed in the presence of a large excess (typically about 5-to about 100-fold) of the tagged, amplified, driver cDNA. Next, those DNA strands which either are themselves tagged or are duplexed with tagged DNA are removed from the mixture. This removal is typically done via exposure of the mixture of DNA strands to a resin or matrix which has affinity for the tag used on the primers earlier. In a preferred embodiment, magnetic beads coated with streptavidin are used. Other resins, such as streptavidin agarose could be used in conjunction with a biotin tag. Tagged single-stranded or duplex cDNA will be retained on the affinity resin, and the non-tagged species, which are not retained, can be found in the flowthrough or supernatant. In this technique, the cDNA from the control cell population is “subtracted” from the cDNA of the target cell population. The remaining, non-tagged cDNA library is said to be “enriched”. The remaining, non-tagged cDNA sequences are then again amplified by means of the polymerase chain reaction with non-tagged primers. After amplification of the remaining non-tagged cDNA sequences, the nontagged cDNA library is again heated and reannealed in the presence of a large excess (typically about 5-to about 100-fold) of the original tagged cDNA library. Removal of all tagged DNA molecules and reamplification of remaining tagged sequences again follows. The combination of steps involving heating and reannealing, removed tagged molecules, and reamplifying remaining, non-tagged molecules constitutes one round. The methods of the present invention involve repeating the rounds from zero to many times. In a preferred embodiment, the method involves a total of approximately 3 to 5 rounds.

In a particularly preferred embodiment, the method involves performing the steps as described above in parallel with a second set of steps in which the cDNA library from the target population of cells is instead subtracted from the cDNA library from a control population. This means that in the second set of steps, the cDNA library from the target cell population is amplified with tagged primers and the cDNA library from the control cell population is amplified with non-tagged primers. The original cDNA of the target cell population is repeatedly subtracted from the cDNA of the control cell population, and separately, the original cDNA of the control cell population is repeatedly subtracted from the target cell population. In the final round of the preferred embodiment of the method, one of the two enriched cDNA libraries obtained from the two sets of steps is subtracted from the other enriched EDNA library. Other alternative methods can be found in Molecular Cloning: A Laboratory Manual, 2nd. ed, Vol. 1-3, eds. Sambrook et al., Cold Spring Harbor Laboratory Press (1989).

### Example 2

Once the cDNA library is made, it is used to construct a vector library whereby the carrier vectors are treated, by cutting and splicing, to receive the molecules of cDNA. The choice of vector may vary. However, preferred vectors are virus based vectors.

Especially preferred vectors are pox virus vectors.

Pox Vaccinina Virus (VV) cDNA Library Construction

In one embodiment, a recombinant VV expression library is made by direct ligation (see Scheiflinger F et al 1992 PNAS, M. Merchlinsky & Moss 1992 Virology, M. Merchlinsky et al 1997 Virology). Using this technique, a unique site restriction site within the VV genome is engineered adjacent to a VV promoter. The VV genome is digested at the unique restriction site to make two vector arms, taking care not to shear the large DNA strands. The recombinant gene of interest, with the relevant cloning ends, is ligated with the two vector arms. A marker or drug selection gene may also be co-ligated to aid in recombinant virus identification. The ligated genome is transfected into a non-avian cell line (see Scheiflinger et al 1992) and “rescued” by fowlpox virus. Fowlpox virus will package VV DNA and thus allow production of replication competent recombinant virus.

Wild type FPV will not replicate in non-avian cells. This technique may suffer from the disadvantage that ligation of a cDNA library into VV by direct ligation may be a very inefficient process due to the large size of the VV genome of 180-200 kb compared with the size of the vector arms of Lambda phage of about 35 Kb.

### Example 3

Minimal Vaccinia Viral (VV) Genome

To improve ligation efficiency the poxvirus genome size can be reduced. Average pox genome is approx 200 kb, compared to Lambda ˜35 Kb. It has been shown that large areas of the VV genome are non-essential for virus replication. In another embodiment, the size of the VV genome is decreased in order to enhance the efficacy of making cDNA libraries in VV using the direct ligation system without losing the replicating ability of the virus. A large number of genes within the VV genome have been shown to be non-essential for virus replication. See Moss (1996) review describing VV gene function. Using this information it is possible to delete >40 kb of the genome.

Accordingly a minimal pox virus based vector can be created whereby there is either

(i) specific deletion of so called non-essential genes or natural deletions of VV during repeated passage in cell culture may also be a method to minimise the genome (e.g. Meyer et al., 1991, J Gen Virol, 72: 1031)

Constructing a Minimal Vaccinia Viral (VV) Genome.

During the process of deleting specific VV genes, stable integration of marker genes cannot be used as this will only add to the size of the VV genome. However, marker/reporter genes are required during the process so viruses that possess genomes with deleted genes can be isolated. Recombinant VV (rVV) transfer plasmids have been developed that, during the initial recombination a selection gene is transiently inserted into the rVV genome. However, when the drug selection is reversed the virus genome will be prone to a second recombination event which results in deletion of the selection or marker gene from the rVV genome.

Such rVV transfer plasmids are modified to contain ˜300 bp of DNA derived from either side of the target gene to be deleted. After recombination the target gene is deleted but the virus will transiently express the selection/marker gene. When drug selection is taken away the selection gene will recombine out. Additionally, those viruses not expressing the marker gene can be isolated.

The approach is used to delete large fragments of the VV genome e.g. the 21 kb non-essential region located at the left hand side of the genome (Hind III C-M). Alternatively, the selection gene can be co-transfected with the recombining plasmid to enrich for recombinant progeny (Kurilla 1997, Isaacs 1990, Scheiflinger et al 1998).

Targeted deletion of non-essential genes is greatly helped by the availability of the genome sequence of several strains of vaccinia virus (see Johnson et al 1993). Typically, genes described as immune evasion molecules can be deleted as these genes are generally not essential for replication in vitro e.g. C3L, K3L, E3L, B8R, B14R, B16R, B19R, G2R, A44L (see Moss 1996). In addition, certain ORFs that express proteins that are extra cellular enveloped virus specific e.g. F13L, A34R, A36R, A56R, B5R, as not essential for virus replication in vitro (see Moss 1996).

Perkus et al (1989) et al studied the effects of a VV that had a 21.7 Kb beginning 3.8 kb from the left hand end of the VV genome. The mutant virus vP293 is still able to replicate in Chicken embryo fibroblast cells. However, restoration of the KIL gene enables the virus to replicate in human cells.

Perkus et al (1991) reported that deletion of 55 open reading frames from the termini of vaccinia virus resulted in a virus that was still replication competent. Each copy of the inverted terminal repeat (ITR) of vaccinia virus consists of 8 kb of DNA containing 9 ORFS flanked near the terminus of the genome by 4 kb of repetitive DNA which in turn contains blocks of tandem repeats. Using plasmids containing repetitive DNA as the external arm, Perkus et al generated deletions at both the left and the right ends of the vaccinia genome. The report illustrated engineered deletion within a single vaccinia virus of 32.7 kb of DNA (including 38 ORFS) from the left terminus and 14.9 kb of DNA (including 17 ORFS) from the right terminus.

Although deletion of some genes may render VV replication deficient, other cell types may still be able to support replication e.g. VERO for NYVAC and C7L/K1L mutants (Perkus et al Virology 1990 179:276-286). A report for the construction of an attenuated VV called NYVAC describes the deletion of 17 genes. The virus was still able to replicate in VERO cells. The 17 genes include: J2R, B13R, B14R, A26L, A56R, C7L, C6L, C5L, C4L, C3L, C2L, C1L, N1L, N2L, MIL, M2L, K1L.

### Example 4

Complementing/Helper Cell Lines

Disclosures by Sutter et al (1994) and Holtzer et al illustrate that essential VV genes expressed by engineered cell lines can support defective VV.

Accordingly, in a further embodiment, the size of the VV genome may be further reduced by constructing a helper cell line in which a number of essential VV genes are permanently expressed. Alternatively, a “helper” virus carrying essential VV genes may be used to co-infect target cells to enable replication of the “minimal” VV. Cell lines expressing essential VV genes are known in the art and have been used to propagate VV viruses with deleted essential genes (see Hostzer and Falkner 1997).

### Example 5

Complementing Helper Viruses

Helper viruses could be used to co-express essential genes deleted from the VV mini-genome. The helper virus will express the essential gene products but will not be able to replicate itself. Thus the helper virus will only be present in the host cell. Therefore only recombinant VV genome progeny will be made. An example of a helper virus is an engineered FPV or a semliki Forest Virus (SLFV).

### Example 6

Tri-Molecular Recombination

In a further embodiment, the VV library is constructed using “tri-molecular recombination” as set out in WO 00/28016. Using the “tri-molecular recombination” approach, the library is constructed in a plasmid such that the cDNAs are ligated into a site that is flanked by VV derived DNA. This plasmid library is then transfected into cells along with the VV vector arms (digested in the corresponding VV derived DNA). If recombination occurs the vector arms are joined thus giving rise to a full-length recombinant genome. The genomes are packaged using e.g. FPV.

### Example 7

cDNA Libraries Prepared in Lentivirus Vectors

The cDNA library may also be inserted into a lentivirus vector such as an EIAV lentivirus based system. As lentiviruses can transduce terminally differentiated/primary cells, the lentirvirus based system can be used to select for a DAM in a terminally differentiated/primary target host cell.

### Example 8

Screens

Cell Identification Systems

Identification of host eukaryotic cells infected with a recombinant viral vector and which express the target DAM of the present invention.

The target host cells are reacted with a binding partner. The BP may be an antibody which has been pre-absorbed (by pre-incubating antibody source with the target host cell/cell line) before use in order to remove any cross-reactivity with the target host cell.

### Example 9

Immunostain

An immunostain may be used to detect the reactivity of a binding partner with a DAM of interest. By way of example, a “Live Immunostain” technique is adapted from that described in Earl et al 1998. The sequence of steps in this immunostain technique is set out as follows:

Grow cells on tissue culture dishes that have been pre-treated with agent to improve cell adherence e.g. Concanavalin A

Infect cells with recombinant poxvirus (or retrovirus) library pool (made via modified direct ligation or tri-molecular recombination) at an moi of 0.1-1.0.

Incubate for 10-48 hours

Pour off media and wash several times with PBS plus blocking agent e.g. FCS

Add primary antibody source (use a varying dilutions to determine optimal). Incubate 10 mins to 1 hour

Wash several times

Add secondary anti-species Ab conjugated e.g. to HRP (pre-treat secondary to remove non-specific cross reactivity). An ABC complex may be used to enhance signal.

Wash

Add conjugate substrate

Using a microscope identify stained cell(s) and pick with a sterile implement e.g. toothpick.

Place cells in small volume of media, freeze thaw to release virus

Repeat staining process with released virus. Several repeats may be needed before a homogenous population is produced

PCR cDNA and sequence to identify target antigen (eg TAA of interest)

### Example 10

Sensitive in Vivo Assay for Detecting Target TRV-h5T4.

In this example a specific Ab was used to identify and successfully clone a recombinant VV expressing a target gene. This experiment parallels the screening of a VV library (according to Example 2), produced from cDNA of a cell that is bound by an Ab source, using said Ab reagent. This Ab could be a mAb or a polyclonal Ab.

The assay used in detecting Trv-h5T4 antigen followed a series of steps:

Grow cells (e.g. CEF cells) in 6 well tissue culture plates until they reach cell density of approximately 1×10⁶ cells per well (e.g. a near confluent monolayer of CEF cells). The exact cell number is important as it is used to calculate the MOI of the virus stocks.

Fresh sucrose purified MVA wt (p581) and Trv-h5T4 were used to prepare virus sock solutions of 1×10⁶ pfu/ml for each virus, maintained in MEM growth medium. These viral stocks are used as a source material for titration of each virus and making up each MOI stock (i.e. diluent for MVA and Trv-h5T4 co-infection). Trv-h5T4 was serially diluted using MVA wt (in 2% MEM medium) as diluent. By way of example: 300 μl of Trv-h5T4 in 2.7 ml of MVA wt containing 1×10⁵ pfiu/ml (MOI=0.1), 2×10⁵ pfu/ml (MOI=0.2), and 3×10⁵ pfu/mp (MOI=0.3) respectively.

Trv-h5T4 virus was titred by immunostaining at 24 hrs post-infection with anti-h5T4 mAb (H8 mAb) supernatant (1/20 dilution) and Rab anti-mouse HRP (1/1000 dilution) in the presence of DAB solution substrate. MVA wt was titred with Rab anti-Vacc (1/1000 dilution) and goat anti-Rab HRP (1/1000 dilution) in the presence of DAB (for results of the sensitivity assay see Table 1).

Results from ‘Sensitivity Assay’

A recombinant vaccinia virus, based on MVA, expressing the oncofoetal TAA h5T4 (TroVax) antigen was used to spike a pool of wild type MVA at a ratio 1:5×10⁶ pfu.

Using live immunostaining procedure (according to the technique described in Example 9) cells infected by TroVax were isdentified. Positive cells were isolated and re-cloned until a homogenous virus isolate was produced. The cDNA of this isolate could then be sequenced using primers based on the flanking sequences of the site into which the library has been cloned.

Thus the sensitivity assay, can detect and propagate (amplify/plaque purify) a single pfu of TroVax in a population of at least 415,000 pfu of MVA wild type (see Table 1).

TABLE 1
Demonstrates that a single pfu of recombinant MVA-h5T4 can be detected
in a population of at least 415,000 pfu of MVA wild-type virus

| | | | | | MVA wt | MVA wt | |
|---|---|---|---|---|---|---|---|
| | | | | | and | and | |
| | | | | Trv- | Trv-h5T4 | Trv-h5T4 | |
| | | MVA wt | | h5T4 | co- | co- | |
| | | only | | only | infection | infection | |
| | | titre | | titre | Trv-h5T4 + | Trv-h5T4 + | |
| MOI | | MVA wt | | Trv-h5T4 | foci | foci | Assay |
| for | MVA wt | Titred | Trv-h5T4 | Titred | observed | observed | Sensitivity |
| MVA | Predicted | pfu/well | Predicted | pfu/well | pfu/well | pfu/well | Detection |
| infn | pfu/well | (Avg 4 wells) | pfu/well | (Avg 4 wells) | LIVE | FIXED | Limit |
| 0.1 | 1 × 10⁵ | 1.25 × 10⁵ | 10 | 9.25 | Well A = 4 | Well A = 7 | |
| | | | | | Well B = 4 | Well B = 6 | |
| | | | 1 | 0.75 | ₁Well A = 1? | Well A = 0 | |
| | | | | | ₂Well B = 1 | Well B = 0 | 1:125,000 |
| 0.2 | 2 × 10⁵ | 2.50 × 10⁵ | 10 | 9.25 | Well A = 1 | Well A = 6 | |
| | | | | | Well B = 3 | Well B = 2 | |
| | | | 1 | 0.75 | ₃Well A = 1 | Wcll A = 0 | |
| | | | | | _{4,5}Well 9 = 2 | Well B = 0 | 1:250,000 |
| 0.3 | 3.33 × 10⁵ | 4.15 × 10⁵ | 10 | 9.25 | Well A = 2 | Well A = 3 | |
| | | | | | Well B = 0 | Well B = 1 | |
| | | | 1 | 0.75 | Well A = 0 | Wcll A = 1 | |
| | | | | | ₆Well 9 = 1 | Well B = 0 | 1:415,000 |

Viability Assay of ‘Live’ h5T4 Encoding Recombinant MVA.

The viability assay was set out as follows:

Foci were generated following co-infection of CEF cells with MVA wt and Trv-h5T4 viruses (according to the Sensitivity assay). Foci harbouring recombinant MVA-h5T4 virus and expressing h5T4 antigen were identified by in vivo imrnunostain (according to the technique described in Example 9). In total six positive foci were picked and transferred to 1 ml cryovial in 2% MEM growth medium.

Samples were freeze-thawed three times in order to release virus which was then used to re-infect 6 well tissues culture plates containing confluent CEF cells for recombinant MVA-hST4 virus expansion.

Newly infected cells were harvested 6 days post-infection when maximum CPE is observed.

Cells were tested for viability of MVA-h5T4 by plating cell lysate harvest on 6 well tissue culture pates containing confluent CEF cells at 10-2 to ₁₀4 dilutions and immunostaining with H8 mAb at 24 hours post infection.

Results from ‘Viability Assay’

Of the six ‘live’ picks assayed only one pick was shown to be non-viable. Thus 5 out of 6 picks contained viable recombinant MVA-h5T4 virus. Referring to the data presented in Table 1, the foci (numbered 1-6 and marked in bold font) were assayed for viability as described and the results are presented in Table 2.

TABLE 2
Demonstrates that of the six ‘live’ foci that were picked, only one was
non-viable i.e. 5/6 of the foci contained viable recombinant
MVA-h5T4 virus.

| ‘Live’ Foci | | | h5T4 + ive |
|---|---|---|---|
| Pick # | Original MOI | Well | foci detected |
| 1 | 0.1 | A | No |
| 2 | 0.1 | B | Yes |
| 3 | 0.2 | A | Yes |
| 4 | 0.2 | B | Yes |
| 5 | 0.2 | B | Yes |
| 6 | 0.3 | B | Yes |

### Example 11

Identification by MoFlo

This technique is substantially the same as the immunostain technique but the following steps are used:

use secondary anti-species Ab conjugated to a fluorescent marker e.g. FITC.

Trypsinise cells after addition of the secondary (Alternatively, use a suspension cell and carry out staining process as for FACS protocol)

Use a MoFlo fluorescent cell sorter to identify those cells with a fluorescent tag.

Release virus from positive cells by freeze thaw

Repeat screening steps with released virus and identify gene as for above

### Example 12

Identification by MACS (Magnetic Beads): System to Select for Antibody Bound Cells.

The antibody staining is carried out as indicated in Example 9. However, the secondary antibody is added whilst cells are in suspension. The secondary Ab is conjugated to a metal bead. A magnet is used to select for the positive cells. The reagents and protocols used are based on the MACS cell separation system (Miltenyi Biotech, Germany).

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry or biology or related fields are intended to be covered by the present invention. All publications mentioned in the above specification are herein incorporated by reference.

### REFERENCES

Carroll, M W, G W Wilkinson & K Lunstrom, 2001 Mammalian expression systems and vaccination Genetically Engineered Viruses Ed C J Ring & E D Blair pp107-157 BIOS Scientific Oxford UK

Earl, P, L. S. Wyatt, B. Moss & M. W. Carroll 1998 Generation of Vaccinia Virus Recombinant Viruses. Current Protocols in Molecular Biology. Supplement 43 Unit 16.17.1-16.17.19. John Wiley & Sons, Inc.

Holzer G W; Falkner F G Construction of a vaccinia virus deficient in the essential DNA repair enzyme uracil DNA glycosylase by a complementing cell line. J Virol 1997 July; 71 (7): 4997-5002

Johnson G P; Goebel S J; Paoletti E An update on the vaccinia virus genome Virology 1993 October; 196(2): 381-401.

Kurilla M G Transient selection during vaccinia virus recombination with insertion vectors without selectable markers. Biotechniques 1997 May; 22(5): 906-10

Merchlinsky, M. et al., 1992, Virology, 190: 522-526

Moss B Poxyiridae: The viruses and their replication In Virology 1996 Ed B N Fields et al Chap 83 pp2637-2671 Lippincott-Raven Publishers. PA USA

Perkus M E; Limbach K; Paoletti Cloning and expression of foreign genes in vaccinia virus, using a host range selection system. J Virol 1989 September; 63(9): 3829-36.

Perkus M E; Goebel S J; Davis S W; Johnson G P; Norton E K; Paoletti E Deletion of 55 open reading frames from the termini of vaccinia virus AUTHOR: Virology 1991 January; 180(1): 406-10 CITATION IDS: PMID: 1984660 UI: 91082435 ABSTRACT:

Scheiflinger, F. et al., 1992, Proc. Natl. Acad. Sci. USA. 89: 9977-9981.

Scheiflinger F; Domer F; Falkner F G Transient marker stabilisation: a general procedure to construct marker-free recombinant vaccinia virus. Arch Virol 1998;143(3): 467-74.

Sutter G; Ramsey-Ewing A; Rosales R; Moss Stable expression of the vaccinia virus K1L gene in rabbit cells complements the host range defect of a vaccinia virus mutant.: J Virol 1994 July; 68(7): 4109-16

Sutter et al (1994) Expressed the K1L gene in RK13 to complement K1L defective VV

Tureci, O, Sahin, U and Pfreundschuh M (1997) Molecular Medicine Today 3: 342-349 Serological analysis of human tumour antigens: molecular definition and implications.

Wyatt, L S, M. W. Carroll, C. P. Czerny, M. Merchlinsky, J. R. Sisler, B. Moss Marker rescue of the host range restriction defects of modified vaccinia virus Ankara. Virology 1998 251:334-42

1. A method for identifying a target disease associated molecule (DAM) comprising:
(i) generating an expression library from DNA or RNA derived from a cell expressing the DAM;
(ii) transfecting the library into a eukaryotic host cell;
(iii) screening the expression library with a screen comprising a binding partner to identify a host cell expressing the target DAM.

2. The method according to claim 1 wherein the target DAM is specific to a cell infected with a virus, fungus, or mycobacteria.

3. The method according to claim 1 wherein the target DAM is specific to an autoimmune disease.

4. The method according to claim 1 wherein the target DAM is a tumour associated antigen (TAA).

5. The method according to any one of claims 1-4 wherein the expression library is constructed in a virus based vector.

6. The method according to claim 5 wherein the viral vector is a pox viral vector.

7. The method according to claim 6 wherein the viral vector is a vaccinia-viral vector.

8. The method according to claim 7 wherein the pox-viral vector is a entomopox viral vector.

9. A method for identifying a target DAM comprising:
(i) producing a cDNA library of a target cell;
(ii) inserting the cDNA library into a virus based vector with a minimal viral genome;
(iii) transfecting the vector into a eukaryotic host cell to produce a transfected host cell;
(iv) culturing the host cell to express the target DAM;
(v) contacting a sample containing a binding partner for the target DAM with a sample of lysed, non-transfected host cells to remove any binding partner from the sample which is specific for non-transfected host cell, to produce a stripped sample;
(vi) contacting the stripped sample to a sample of lysed host cells transfected with the same vector into which the cDNA has been inserted wherein the vector does not contain any cDNA, to remove any binding partner specific for said vector, thereby producing a twice stripped sample;
(vii) contacting the twice stripped sample to the lysate of (v), whereby any binding partner specific for the binding partner binds thereto; and
(viii) determining binding in (viii) to identify the target DAM.

10. The method according to claim 9 wherein the method further comprises identifying the transfected host cell which expressed the target DAM and isolating the cDNA contained therein.

11. The method according to claim 9 wherein the method further comprises isolating and/or purifying a specific binding partner for the target DAM.

12. The method according to any one of claims 9-11 wherein the virus based vector with a minimal viral genome is a pox viral vector.

13. The method according to claim 12 wherein the viral vector is a vaccinia-viral vector.

14. The method according to claim 13 wherein the pox-viral vector is an entomopox viral vector.

15. The method according to any one of claims 9-14 wherein the target cell is a cancer cell.

16. The method according to any one of claims 9-15 wherein the binding partner is an antibody.

17. The method according to any one of claims 9-16 wherein the sample is serum.

18. The method according to any one of claims 9-17 wherein the target DAM is a TAA.

19. An antigen identified by the method according to any one of claims 9-18.

20. An antigen according to claim 19 for use in medicine.

21. Use of an antigen according to claim 19 or claim 20 in the manufacture of a medicament for the treatment of a disorder associated with a DAM.

22. A method for identifying a target disease associated molecule (DAM) comprising:
i) generating an expression library from DNA or RNA derived from a cell expressing the DAM;
ii) transfecting the library into a eukaryotic host cell;
iii) screening the expression library with a screen comprising an agent to identify a host cell expressing the target DAM;

23. A method for identifying a target disease associated molecule (DAM) according to claim 22 wherein the agent is an antibody.

24. A method for identifying a target disease associated molecule (DAM) according claim 23 wherein the antibody is a polyclonal antibody.

25. A method for identifying a target disease associated molecule (DAM) according to claim 23 wherein the antibody is a monoclonal antibody.
